(19) United States
(12) Patent Application Publication
(10) Pub. No.: US 2016/0115538 A1
(43) Pub. Date: Apr. 28, 2016

(54) Methods and Systems for Using Photoswitchable Nucleic Acids to Control Hybridization Stringency

(71) Applicant: UNIVERSITY OF WASHINGTON THROUGH ITS CENTER FOR COMMERCIALIZATION, Seattle, WA (US)

(72) Inventors: David GINGER, Seattle, WA (US); Yunqui YAN, Seattle, WA (US); Jennifer I-Ling CHEN, Toronto (CA)

(73) Assignee: University of Washington through its Center for Commercialization, Seattle, WA (US)

(21) Appl. No.: 14/889,575

(22) PCT Filed: May 9, 2014
(86) PCT No.: PCT/US2014/037557

§ 371 Date: Nov. 6, 2015

Related U.S. Application Data
(60) Provisional application No. 61/821,638, filed on May 9, 2013.

(51) Int. Cl. C12Q 1/68; G01N 21/47; G01N 21/64; G01N 21/31

(52) U.S. Cl. CPC C12Q 1/6883; G01N 21/3103; G01N 21/47; G01N 21/648; C12Q 2600/156

(57) ABSTRACT

Compositions, methods and systems are provided that enable light-controlled hybridization between two nucleic acid sequences and further enable the characterization of one or more sequence variations between the nucleic acids.

### CROSS-REFERENCE TO RELATED APPLICATION

This application claims the benefit of U.S. Patent Application No. 61/821,638, filed May 9, 2013, the disclosure of which is hereby incorporated by reference in its entirety.

### STATEMENT OF GOVERNMENT LICENSE RIGHTS

This invention was made with government support under FA9550-10-1-0474, awarded by the Air Force Office of Scientific Research, and CMMI 0709131, awarded by the National Science Foundation. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 44034_SEQ_ST25.txt. The text file is 4 KB; was created on May 9, 2014; and is being submitted via EFS-Web with the filing of the specification.

### BACKGROUND

A great number of genes that influence or underlie diseases have been identified. Genetic bases for more conditions are continually being discovered. Therefore, detection of genetic variations is crucial for detecting predispositions for such diseases. Accordingly, it is desirable to develop simple, sensitive techniques to reliably identify and characterize sequence variations.

The hybridization between complementary nucleic acid sequences has been used to analyze sequence variations in test populations. Hydrogen bonding between complementary bases in DNA leads to the hybridization of two strands into a duplex structure. Conventionally, thermal energy such as heat, or changes in ionic strength (salt gradients), are required to melt (dehybridize) the two strands when performing analytical techniques, such as hybridization stringency washes. However, temperature and concentration gradients can be difficult to control precisely in the context of such automated solution-based assays, which may hinder precision. Furthermore, any sequence variations between the two strands affect the melting threshold for the duplex structure by affecting the stability of the hybridization, thereby further hindering precision of assays.

Therefore, a need remains for alternative approaches to detect sequence variations in light of the various conditions that can influence hybridization of nucleic acids detection assays. The present disclosure presents improved approaches to address these needs and provide additional benefits.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a system is provided. In one embodiment, the system comprises a first nucleic acid comprising a photoswitchable molecule and a probe sequence, wherein the photoswitchable molecule is capable of undergoing a structural change from a first conformation to a second conformation upon illumination by a first wavelength of light at a first photonic energy, wherein the structural change alters a hybridization property of the first nucleic acid sequence in relation to a target sequence. In one embodiment, the system also comprises a second nucleic acid comprising the target sequence; wherein the target sequence is partially complementary to the probe sequence, such that the target sequence is configured to hybridize with the probe sequence; and wherein there is a base-pair mismatch between the target sequence and the probe sequence at a position four or fewer bases away from the photoswitchable molecule. In one embodiment, the system also comprises liquid media providing liquid communication between the first nucleic acid and the second nucleic acid.

In another aspect, a method of detecting a sequence variation in a nucleic acid is provided. In one embodiment, the method comprises providing a first nucleic acid comprising a photoswitchable molecule and a probe sequence, wherein the photoswitchable molecule is capable of undergoing a structural change from a first conformation to a second conformation upon illumination by a first wavelength of light. In one embodiment, the method also comprises contacting the first nucleic acid with a second nucleic acid comprising a target sequence that is at least partially complementary to the probe sequence, wherein the first nucleic acid is contacted with the second nucleic acid under conditions that permit the target sequence to hybridize to the probe sequence, and wherein the photoswitchable molecule is incorporated into the first nucleic acid at a position four or fewer bases away from the nucleotide position in the probe sequence that hybridizes to the position on the target sequence with a suspected sequence variation. In one embodiment, the method also comprises applying a first wavelength of light at a first photonic energy, thereby promoting a structural change in the photoswitchable molecule that alters a hybridization state of the probe sequence in relation to the target sequence. In one embodiment, the method also comprises monitoring the hybridization state of the probe sequence in relation to the target sequence, wherein a conversion to a destabilized, hybridized state or to an unhybridized state between the probe sequence and the target sequence indicates the presence of a sequence variation in the target sequence compared to the probe sequence.

In another aspect, a method for designing a probe for detecting a sequence variation in a nucleic acid is provided. In one embodiment, the method comprises obtaining the sequence of a reference nucleic acid, or a complement thereof; determining the location in the reference nucleic acid sequence, or the complement thereof, of a suspected sequence variation; and designating in the reference nucleic acid sequence, or the complement thereof, at least one position within four nucleic acid positions of the location of the suspected sequence variation to receive the incorporation of a photoswitchable molecule, wherein the photoswitchable molecule is capable of undergoing a structural change from a first conformation to a second conformation upon illumination by a first wavelength of light.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C. (FIG. 1A) Absorption spectra of free trans-azobenzene in isooctane and azobenzene-modified ssDNA in buffer. The structure of free azobenzene in the trans form is shown in the inset. (FIG. 1B) Structure of azobenzene-modified DNA with the d-threoninol linkage, adenine, and cytosine labeled. (FIG. 1C) DNA nucleotide structures of the abasic site, thymine, and guanine used in the sequences and described in the disclosure.

FIGS. 2A-2B. (FIG. 2A) Representative plots of the measured fraction of cis-azobenzene vs. the integrated photokinetic factor (see Equation 2 below) used to obtain quantum yield. Solid lines are fits of Equation 1 to the data shown, and are labeled with the average quantum yield values measured from at least three separate experiments. Traces are for azobenzene in isooctane (circles), azobenzene incorporated in ssDNA (SEQ ID NO:1 with an azobenzene represented as “X” between nucleotides 9 and 10; squares) and dsDNA (SEQ ID NO:1 and its complement, SEQ ID NO:2; triangles). (FIG. 2B) Similar plots for azobenzene incorporated in different dsDNA sequences including Seq-mm-abasic (circles), Seq-mm1T (squares), Seq-mm2 (triangles) and Seq-complement (diamonds) (see Table 1, below, for sequences).

FIG. 3. The trans-to-cis isomerization quantum yield plotted as a function of melting temperature (Tₘ) of azobenzene-modified dsDNA. Quantum yield decreases as Tₘ rises. The quantum yield is very sensitive to the single-base mismatch at the nearest neighbor position of the azobenzene (circles), but is less sensitive for dsDNAs having the mismatched base multiple bases away from the azobenzene (triangles and the square).

FIG. 4. The quantum yield (bars) for the azobenzene incorporated into the 18-base ssDNA of poly(dC), poly(dA), and poly(dT). The oxidation potential for the individual DNA nucleotides (C, A, T) vs. saturated calomel electrode (SCE) is also plotted (dots). There does not appear to be a correlation between the oxidation potential for the nucleotides and the quantum yield of the azobenzene incorporated into the ssDNA sequences, as might be expected if charge transfer was the dominant factor governing azobenzene quantum yield differences between the sequences.

FIGS. 5A-5B. The trans-to-cis isomerization quantum yield explains photon-dose-controlled DNA hybridization stringency wash. (FIG. 5A) DNA sequences used to incorporate azobenzenes and also to crosslink gold nanoparticles. “Sequence 1” is set forth in SEQ ID NO:10 and has azobenzenes indicated with “X” inserted between nucleotides as positions 11 and 12, 13 and 14, 15 and 16, and 17 and 18. “Sequence 2” is set forth in SEQ ID NO:11. “Sequence 3, Seq-perfect” is set forth in SEQ ID NO:12. “Sequence 3, Seq-mmT” is set forth in SEQ ID NO:13. “Sequence 3, Seq-mmA” is set forth in SEQ ID NO:14. “Sequence 3, Seq-mmC” is set forth in SEQ ID NO:15. (FIG. 5B) The quantum yield of the azobenzene is shown against each embedding dsDNA. The inset in FIG. 5B is a plot of the gold nanoparticle localized surface plasmon resonance (LSPR) peak position as a function of the photon dose. The azobenzene photoisomerization quantum yield increases from Seq-perfect, to Seq-mmT, to Seq-mmA and to Seq-mmC. Nanoparticle conjugates linked by these dsDNA show the same increasing order of photoinduced disaggregation rate.

FIG. 6. Schematic illustration of relationship between proximity of base-pair mismatch to azobenzene and the detected quantum yield upon illumination.

FIGS. 7A-7B. Plots of fraction of cis-azobenzene as a function of UV excitation time before converting excitation time to the integrated photokinetic factor (in FIGS. 2A-2B). The fraction of cis-azobenzene increases and reaches the photo-stationary state value within 10-20 min after UV exposure.

FIG. 8. An example plot of the absorbance at 260 nm as a function of temperature to obtain the melting temperature (Tₘ) of azobenzene-modified dsDNA. The Tₘ is determined as the temperature at which the first derivative of absorbance against temperature is the largest. The plot shown here corresponds to the dsDNA of Seq-complement with Tₘ equals to 60° C.

FIG. 9. Absorption coefficient spectra of DNA nucleotides in buffer (pH=6.5) and trans-azobenzene. The spectra show that DNA nucleotides absorb photon energy mainly between 240-280 nm while the trans-azobenzene absorbs between 300-350 nm. There is little overlap in the transition band between DNA nucleotides and the trans-azobenzene.

FIGS. 10A-10B. Experimental results exclude the influence of UV-vis absorption measurement on azobenzene isomerization. Fraction of cis-azobenzene is plotted as a function of absorption measuring time for free azobenzene in isooctane. (FIG. 10A) Azobenzene started in trans form. The fraction of cis-azobenzene was almost zero after intense absorption spectrum was taken every half min for 1 h, proving that trans-azobenzene does not isomerize to cis-azobenzene after exposure to the light source of a UV-vis spectrometer. (FIG. 10B) Azobenzene started in cis form after being converted from trans form by UV excitation. The absorption spectra were measured every 1 min for 1 h. The small decrease in the fraction of cis-azobenzene proves the minimal influence on cis-to-trans isomerization from UV-vis spectrometer light source.

FIG. 11. Schematic illustration of an embodiment of a probe assay configured to detect two distinct single nucleotide polymorphisms, each at a different location in the source DNA (e.g., different locations in the same gene).

### DETAILED DESCRIPTION

Compositions, methods and systems are provided that enable light-controlled hybridization between two nucleic acid sequences and the characterization of sequence variations in target nucleic acids.

In one aspect, a system is provided. In one embodiment, the composition includes:

a first nucleic acid comprising a photoswitchable molecule and a probe sequence, wherein the photoswitchable molecule is capable of undergoing a structural change from a first conformation to a second conformation upon illumination by a first wavelength of light at a first photonic energy, wherein the structural change alters a hybridization property of the first nucleic acid sequence in relation to a target sequence;

a second nucleic acid comprising the target sequence, wherein the target sequence is partially complementary to the probe sequence, such that the target sequence is configured to hybridize with the probe sequence, and wherein there is a base-pair mismatch between the target sequence and the probe sequence at a position four or fewer bases away from the photoswitchable molecule; and

liquid media providing liquid communication between the first nucleic acid and the second nucleic acid.

Specifically, the system incorporates the photoswitchable molecule into the structure of a nucleic acid molecule. The general incorporation of photoswitchable molecules into nucleic acids is described in U.S. Application No. 2010/0143331, incorporated by reference herein in its entirety. In some embodiments, the photoswitchable molecule is incorporated into the first nucleic acid molecule. The photoactive properties of the modified first nucleic acid are then utilized to control the hybridization state of the first nucleic acid (and/or a probe sequence therein) with the second nucleic acid (and/or a target sequence therein). The modification of the hybridization state, such as causing the transition from a stable, hybridization state to a dehybridization state, or vice versa, including transitions to destabilized intermediate states where the first nucleic acid molecule and the second nucleic acid molecule are still hybridized, is useful for detecting and identifying sequence variations in a nucleic acid. This is accomplished by virtue of detecting or identifying sequence mismatches between the nucleic acids that occur within four nucleotide positions of the photoswitchable molecule by carefully manipulating the hybridization state of the duplex by control of the photoisomerization of the photoswitchable molecule incorporated in the duplex.

As used herein, the term “nucleic acid” refers to DNA (deoxyribonucleic acid) or RNA (ribonucleic acid), and variants thereof such as synthetic variants. Nucleic acids are synonymous with polynucleotides. Nucleic acids molecules can be single stranded or double stranded (with complementary single-stranded polynucleotide chains hybridizing by base pairing of the individual nucleobases). However, unless explicitly described otherwise, the terms “first nucleic acid” and “second nucleic acid” are generally used herein to refer to single stranded nucleic acid molecules.

The term “nucleotides” refers to the individual subunits of the nucleic acid polymers. A nucleotide is composed of a nucleobase, a five-carbon sugar (either ribose or 2-deoxyribose), and one or more phosphate groups. By virtue of the covalent bonding of the sugar of one nucleotide to the phosphate of another nucleotide, a plurality of nucleotides are joined in a polynucleotide chain with an alternating sugar-phosphate backbone. The nucleotides at each position on the polynucleotide chain can have a distinct nucleobase (or no base at all, termed an “abasic” site or residue), thus providing a particular sequence to the nucleic acid. As is known in the art, the canonical nucleobases for DNA are guanine (G), adenine (A), thymine (T), or cytosine (C). The canonical nucleobases for RNA are guanine (G), adenine (A), uracil (U), or cytosine (C). In terms of DNA, two opposing single stranded polynucleotides can hybridize in a double stranded configuration wherein hydrogen bonds are formed between complementary nucleobases. The hydrogen bonds contribute to the stability of the hybridized duplex. The purine nucleobase, adenine (A), typically forms two hydrogen bonds with the pyrimidine nucleobase thymine (T), and the purine nucleobase, adenine (A), typically forms three hydrogen bonds with the pyrimidine nucleobase cytosine (C). When an RNA polynucleotide participates in a double stranded hybridization, adenine (A), typically forms two hydrogen bonds with uracil (T). Any alignment (e.g., hybridization) of two polynucleotides with the canonical nucleobases that results in the alignment of nucleobases other than the above pairings is considered to be a “base-pair mismatch” (e.g., a G aligned with a T instead of a C is a base-pair mismatch) and is indicative of a sequence variation as between the two nucleic acids.

The nucleic acids of the present disclosure can also include synthetic variants of DNA or RNA. “Synthetic variants” encompasses nucleic acids incorporating known analogs of natural nucleotides/nucleobases that can hybridize to nucleic acids in manner similar to naturally occurring nucleotides. Exemplary synthetic variants include peptide nucleic acids (PNAs), phosphorothioate DNA, locked nucleic acids, and the like. Modified nucleobases can include, but are not limited to, 5-Br-UTP, 5-Br-dUTP, 5-F-UTP, 5-F-dUTP, 5-propynyl dCTP, 5-propynyl-dUTP, and the like. Persons of ordinary skill in the art can readily determine what base-pairings for each modified nucleobase are deemed a base-pair match versus a base-pair mismatch.

Furthermore, the term “sequence” with reference to any nucleic acid molecule, refers to a plurality of adjacent, covalently-linked nucleotides, which may constitute an entire nucleic acid molecule or a sub-portion thereof. Unless otherwise indicated, reference to a particular nucleic acid sequence includes the complementary sequence thereof, which can be determined by virtue of the known complementary base-pairings, described above. In some instances herein, a sequence is referred to as a “probe sequence,” which refers to the sequence in a nucleic acid (e.g., a first nucleic acid) that is used to detect or identify potential variations in the sequence of a target nucleic acid (e.g., “second” nucleic acid) that is suspected of having a sequence variation at one or more nucleotide positions. Accordingly, in other instances herein, a sequence is referred to as a “target sequence,” which refers to the sequence in a nucleic acid (e.g., a second nucleic acid) that is suspected of having at least one sequence variation with respect to the reference or probe sequence. In some embodiments, the probe or reference sequence can be considered the wild-type sequence. In some embodiments, the target sequence is considered the unknown sequence that is being assayed for variation from the reference. The nucleic acid sequences referred to herein can be sequences on separate nucleic acid chains (e.g., one sequence on each strand of double-stranded DNA) or on a single nucleic acid chain (e.g., RNA that is folded over onto itself so as to arrange the two different sequences in close proximity).

The term “partially complementary” is used to describe the relationship between the probe sequence and the target sequence, wherein at least one base-pair mismatch exists between the probe sequence and the target sequence when the first nucleic acid and the second nucleic acid are aligned for optimal hybridization. The at least one base-pair mismatch indicates a sequence variation between the probe sequence and the target sequence (considering their respective complement sequences). A base-pair mismatch can include an abasic site, a modified base that does not form equivalent hydrogen bonds as the base-pair match, or a combination thereof. Notwithstanding one or more base-pair mismatches between the probe sequence and the target sequence, the probe sequence and the target sequence incorporate a sufficient level of complementarity (e.g., number of complementary nucleobases at corresponding sites) that the first nucleic acid (or probe sequence) and the second nucleic acid (or target sequence) can specifically hybridize under appropriate conditions, which are readily determined by skilled persons in the art. In some embodiments, the structural change conferred by the base-pair mismatch destabilizes hybridization of the first nucleic acid (or probe sequence) with the second nucleic acid (or target sequence). This is considered to be an intermediate hybridization state, wherein the nucleic acids are hybridized but destabilized, and the destabilized hybridization requires an amount of photonic energy that is less than an amount of photonic energy at the same wavelength as required to destabilize hybridization of the first nucleic acid with the second nucleic acid to the same extent if they were more complementary (e.g., had fewer or no base-pair mismatches). That is to say that the greater the extent of mismatch, the less photonic energy required to destabilize hybridization.

A “photoswitchable” molecule is one that changes conformations when illuminated with electromagnetic radiation (e.g., light) at a first photonic energy. In certain embodiments, the photoswitchable molecule photoisomerizes from a first conformation to a second conformation, such as from a cis conformation to a trans conformation or vice versa. In certain embodiments, the photoswitchable molecule is reversibly photoswitchable, such that a first wavelength of light changes the conformation of the molecule from a first state to a second state; and a second wavelength of light reverses the conformation change from the second state back to the first state. A representative photoswitchable molecule is azobenzene (and photoswitchable analogs thereof). Further representative photoswitchable molecules include other azobenzenes, stilbenes, spiropyrans, fulgides, diarylethenes, diphenylpolyenes, dihydro-indolizines, diarylethanes, chromenes, napthopyrans, spiropyrans, fulgides, fulgimides, spiroxazines, photoswitchable analogs thereof, and/or any other compounds known in the art that undergo structural changes upon photoexcitation. Persons of skill in the art can readily ascertain the appropriate wavelength, or range of wavelengths, that cause or promote the changes in conformation for each of the above photoswitchable molecules.

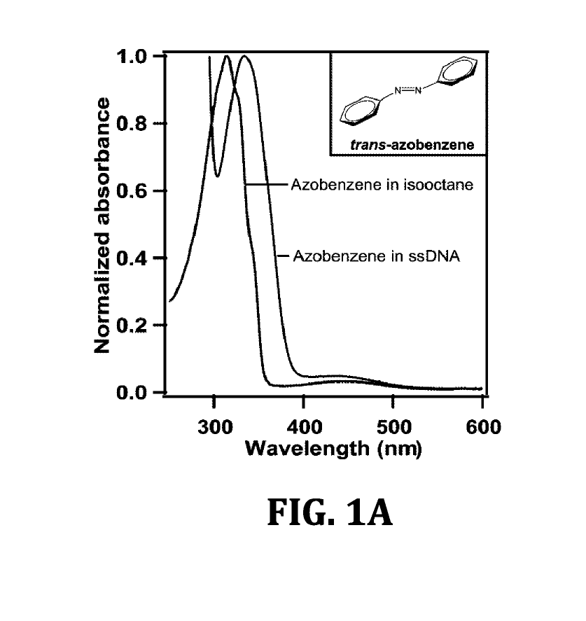

In some embodiments, the photoswitchable molecule is incorporated into the first nucleic acid. In some embodiments, such incorporation is by covalent bond between the photoswitchable molecule and the first nucleic acid sequence (e.g., bound via a base). In some embodiments, the photoswitchable molecule can be linked by covalent bond directly to the sugar-phosphate backbone. For instance, azobenzene (as used in exemplary embodiments herein) molecules are linked by a covalent bond to the nucleic acid sequence, but inserted by intercalation. As described in more detail below, an azobenzene can be incorporated by tethering it to an additional sugar/phosphate linkage along the DNA backbone via a d-threoninol group. Despite some structural distortion of the double helix resulting from the volume of the extra phosphate and azobenzene moieties, the incorporation of a trans form azobenzene stabilizes a DNA duplex by intercalation between the neighboring bases. See, e.g., FIG. 1B. It would be possible for the photoswitchable molecule to be linked by a covalent bond at a site on a nucleic acid sequence that does not allow it to intercalate.

In another embodiment, the photoswitchable molecule is incorporated into the second nucleic acid sequence. In yet another embodiment, there are photoswitchable molecules incorporated into both the first nucleic acid sequence and the second nucleic acid sequence; these photoswitchable molecules may be the same or different on each nucleic acid sequence.

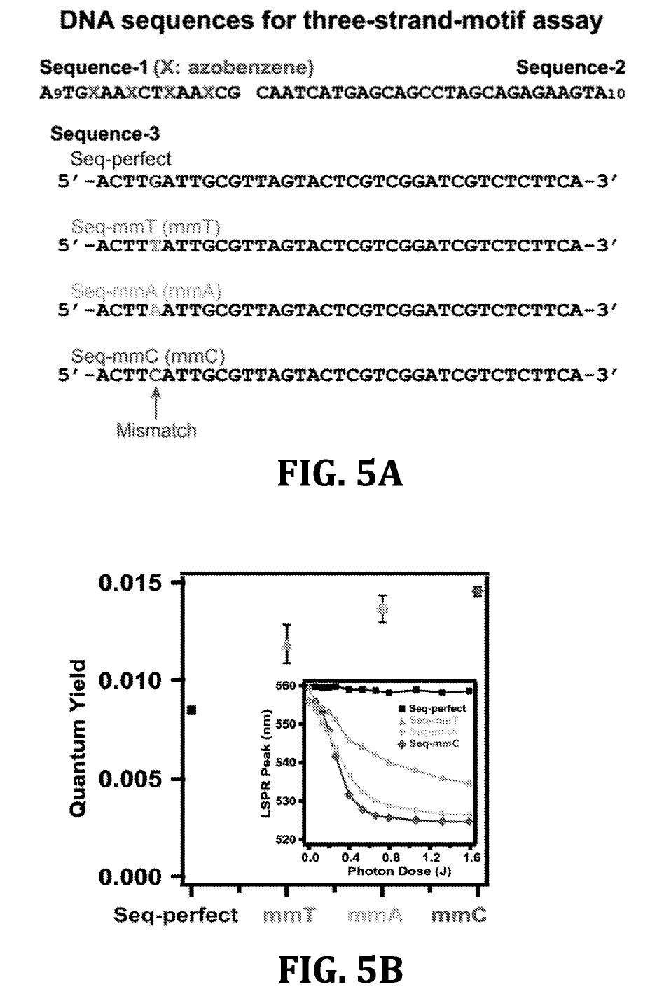

In any of the above embodiments, a photoswitchable molecule is incorporated into the first or second nucleic acid molecule at a position corresponding to a position that is four or fewer bases away from a sequence variation (e.g., a base-pair mismatch). In some embodiments, a photoswitchable molecule is incorporated into the first and/or second nucleic acid molecule at a position that is four or fewer nucleotide positions away from a suspected sequence variation. The term “four or fewer bases away” refers to a base position (also referred to as nucleotide or nucleobase position) that corresponds to a position containing a photoswitchable molecule, wherein the nucleotide/nucleobase position is four, three, two, or one (e.g., adjacent) base positions away from the location of the base-pair mismatch or suspected base-pair mismatch. See, e.g., the scheme in FIG. 6, which illustrates the relationship between the proximity of the photoswitchable molecule to the location of a base-pair mismatch and the detected quantum yield exhibited by photoswitchable molecule within the hybridized nucleic acid construct. It is noted that the term “four or fewer bases away” also encompasses ranges, such one to four, two to four, three to four, one to three, two to three, and one to two base positions between the photoswitchable molecule and the location of a base-pair mismatch when the probe sequence and the target sequence are hybridized. It is noted that the photoswitchable molecule does not need to be incorporated specifically into the probe sequence (or target sequence), but instead merely needs to be at a position that is four or fewer bases away from the base-pair mismatch, wherein the base-pair mismatch is located between the probe sequence and the target sequence. Accordingly, the photoswitchable molecule can be outside the probe or target sequence, but within at least four base positions thereof.

As an illustrative schematic representation of photocontrolled hybridization, FIG. 6 illustrates duplex DNA consisting of a first nucleic acid 104, incorporating azobenzene molecules 102, and a second nucleic acid 106 that is hybridized to the first nucleic acid 102. The azobenzene 102 in the first conformation, a lower energy trans conformation, allows the first nucleic acid 104 and the second nucleic acid 106 to form a relatively stable duplex structure in which the azobenzene 102 molecules intercalate between the DNA nucleobases via π-π stacked interaction. Upon UV irradiation (at a first wavelength of light with a first photonic energy), trans-azobenzene 102 photoisomerizes to the (higher energy) cis-azobenzene 102′, which destabilizes the hybridization of the first nucleic acid and second nucleic acid in the duplex structure. In FIG. 6, it is noted that the cis-azobenzene 102′ is represented by having one of the phenyl rings in dashed lines to indicate the alternate position for this moiety. This change in conformation, thus, can be characterized as causing a conversion of the hybridization state from a stabilized, hybridized state to a destabilized, hybridized state. In some embodiments, the destabilized, hybridized state can be further converted to a dehybridized state altogether by the illumination of a second photonic energy greater than the first photonic energy, by altering other hybridization stringency conditions, as known in the art, or by prolonged illumination at the first photonic energy. Ultimately, the duplex can dehybridize because of changes in the structural conformation of the system induced by the azobenzene and the decrease in the overall energetic stability of the duplex. The reverse isomerization of cis-azobenzene 102′ to trans-azobenzene 102 occurs with blue light irradiation (at a second wavelength, different than the first wavelength); and subsequent cycling of DNA hybridization can be carried out by alternating the light source wavelength.

In one embodiment, the structural change is reversible upon illumination by a second wavelength of light that is different than the first wavelength of light. For example, after UV irradiation, cis-azobenzene can be photoisomerized back to the trans form by irradiating with blue light, thereby allowing or inducing re-hybridization of the probe sequence to the target sequence. In some embodiments, the photoisomerization of cis-azobenzene back to the trans form by irradiating with blue light resulting in a conversion of a hybridization state of the probe sequence and the target sequence from a destabilized, hybridized state to a stabilized, hybridized state. Solutions of first and second nucleic acids can be cycled many times with illumination by a first and second wavelength without noticeable deterioration of the optical properties, suggesting good photostability. The obtained target-induced light modulated optical signal is unique to the disclosed systems and can be used to distinguish target binding from any isotropic background noise.

Accordingly, structural changes in the photoswitchable molecules alter a hybridization property of the first nucleic acid (or probe sequence therein) in relation to a second nucleic acid (or target sequence therein). In one embodiment, alteration in a hybridization property is a destabilization of the hybridization of the probe sequence with the target sequence. In a further embodiment, the alteration in a hybridization property is the dehybridization of the probe sequence from the target sequence. In other embodiments, the alteration in a hybridization property is a stabilization of the hybridization of the probe sequence with the target sequence.

In one embodiment, the first wavelength is a near-infrared wavelength, e.g., any wavelength between about 0.75 μm and about 1.4 μm, or any subrange therein. In one embodiment, the first wavelength is a visible wavelength, e.g., any wavelength between about 390 nm to about 750 nm, or any subrange therein. In one embodiment, the first wavelength is an ultraviolet wavelength, e.g., any wavelength between about 100 nm to about 400 nm, or any subrange therein. As used herein, the term “about” implies a potential variation of 5% above or below the stated value.

For example, in embodiments that incorporate azobenzene as the photoswitchable molecule, the first wavelength of light is between about 280 nm and about 380 nm. For example, in some further embodiments, the first wavelength can be about 280 nm, about 290 nm, about 300 nm, about 310 nm, about 320 nm, about 330 nm, about 340 nm, about 350 nm, about 360 nm, about 370 nm, and about 380 nm, or any subrange therein.

In one embodiment, the first wavelength is less than the second wavelength and the hybridization property is altered to stabilize hybridization of the first nucleic acid sequence with the second nucleic acid sequence.

In one embodiment, the first wavelength is less than the second wavelength and the hybridization property is altered to destabilize hybridization of the first nucleic acid sequence with the second nucleic acid sequence more likely.

In one embodiment, the first wavelength is greater than the second wavelength and the hybridization property is altered to destabilize hybridization of the first nucleic acid sequence with the second nucleic acid sequence.

In one embodiment, the first wavelength is greater than the second wavelength and the hybridization property is altered to stabilize hybridization of the first nucleic acid sequence with the second nucleic acid sequence.

Hybridization states of a first nucleic acid and second nucleic acid, or sequences or subsequences contained therein, can include a stabilized, hybridized state, a destabilized, hybridized state, and an unhybridized state.

As used herein, the term “hybridization property” refers to any characteristic that affects the ability of the first nucleic acid (or probe sequence therein) to hybridize with the second nucleic acid (or target sequence therein). At the extremes, the hybridization property is altered by the photoswitchable molecule to either entirely hybridize the two nucleic acid molecules (e.g., bind together at least their respective probe and target sequences) or entirely dehybridize the two nucleic acids (e.g., remove all binding forces between their respective probe and target sequences). However, in certain embodiments, the photoswitchable molecule only acts to stabilize (i.e., make binding more energetically or entropically favorable) or destabilize (i.e., make binding less energetically or entropically favorable) the hybridization between the nucleic acids.

In situations where the photoswitchable molecule does not completely hybridize or dehybridize the two nucleic acid sequences, other mechanisms can be used to complete the hybridization or dehybridization. For example, a photoswitchable molecule can be used to destabilize hybridization between two bound nucleic acids, without actually dehybridizing them completely, thus achieving a destabilized, hybridized state. This destabilization can manifest itself when the temperature of the bound nucleic acids is raised: the destabilized nucleic acids have a lowered melting temperature (i.e., dehybridization temperature) than if the photoswitchable molecule was not used to destabilize hybridization. Therefore, in certain embodiments, the photoswitchable molecule only contributes to stabilization and/or destabilization of hybridization, with other mechanisms, such as temperature and/or ion concentration, completing the hybridization/dehybridization. Conversely, in one embodiment, the temperature is kept substantially constant during hybridization/dehybridization. In another embodiment, ion concentration is kept substantially constant during hybridization/dehybridization. In another embodiment, a chemical reagent concentration in the local environment is kept substantially constant during hybridization/dehybridization. In this regard, the term “substantially constant” refers to maintenance of the parameter or condition allowing for only a slight variation of up to 5% above or 5% below the parameter value.

In some embodiments, at least the first nucleic acid is attached to the surface. As used herein, “attached” means bound to or otherwise immobilized on the surface. This attachment may be covalent, ionic, electrostatic, or any other mechanism known to those of skill in the art. The first nucleic acid can be directly attached to the surface or can be attached to the surface via a linker (e.g., a portion of the nucleic acid other than the probe sequence). In some embodiments, the second nucleic acid is attached to the surface. In some embodiments, the first nucleic acid is attached to the surface. Finally, in some embodiments, the first nucleic acid and the second nucleic acid are each attached to independent surfaces.

The surface acts as an attachment point for one or more nucleic acids. At least the first nucleic acid is attached to the surface. In certain embodiments, a plurality of nucleic acids (and/or strands) are attached to the surface.

In one embodiment, the surface is a surface of a core, wherein a core is defined as a particle of micro- or nano-scale size, depending on various factors described below. In other embodiments, the surface is a planar surface, such as can be found on an assay chip. Such assay chips are well known to those of skill in the art.

In certain embodiments, the core functions as a reporting structure that can be detected. For example, in certain embodiments, the core is a nano-scale gold particle that exhibits surface plasmon resonance (SPR) such that optical absorbance spectroscopy (e.g., UV-vis) can be used to detect the core in solution. While illustrative optical detection schemes are generally described herein, it will be appreciated that any detection scheme known to those of skill in the art can be used, including electrical or magnetic detection techniques. The composition of the core can be modified as necessary to facilitate the detection technique.

In certain embodiments, the core is a material that has an SPR. When photoswitchable nucleic acids are combined with plasmon-resonant metal nanoparticles, photoswitchable optical properties are created. The photoswitchable optical properties arise from the changes in the plasmon coupling of nanoparticles due to photocontrolled hybridization and dehybridization of the nucleic acids.

In certain embodiments, the core has an external surface that is a metal. Exemplary metals include gold, silver, aluminum, and combinations thereof, including alloys and core/shell particles. In these embodiments, the entire particle may be the single SPR metal, or the SPR metal may only coat a non-SPR metal core, such as a semiconductor or an insulator, as long as the particle as a whole has an SPR or is otherwise detectable as desired. Additional possible core materials include silicon, CdSe, CdS, ZnS, ZnO, polystyrene, latex, Fe₂O₃, CdSe/ZnS core/shell structures, copper, cobalt, platinum, and their respective oxides, and chalcogenides.

In other embodiments, the core is an insulator or semiconductor that does not have an SPR but is useful in an alternative detection scheme, such as by non-SPR optical detection or electrical detection.

In one embodiment, the shape of the core is selected from a sphere, a cylinder, an ellipsoid, a polyhedron, a prism, a rod, and a wire. The shape of the core may contribute to the detection properties, as will be appreciated by those of skill in the art (e.g., nano-rods may have different optical properties than nano-spheres).

In one embodiment, the core has a critical dimension of from 1 nm to 200 nm. The nano-scale size is critical particularly for optical detection techniques (e.g., SPR detection) and to facilitate the reversible aggregation/disaggregation of multiple cores together in a solution (e.g., because larger cores tend to aggregate/adhere to surfaces without complementary DNA). The reversible aggregation/disaggregation occurs in direct result to the hybridization/dehybridization of first and second nucleic acids (or probe and target sequences therein) facilitated by the conformational change of the photoswitchable molecule.

In another embodiment, the core has a critical dimension of greater than one micron. In certain embodiments, such micron-sized cores are formed from polymer or silica.

In one embodiment, the core is optically detectable by changes in absorption, light scattering, or photoluminescence that are triggered by changes in the hybridization state of the first nucleic acid sequence in relation to the second nucleic acid sequence. Furthermore, it is contemplated that the photoswitchable optical properties can be detected by many methods, such as a UV-Vis spectrophotometer, visually by the naked eye as a color change in bulk solution, monitored at the single nanostructure level using a dark-field microscope coupled with a fiber optic spectrometer, or detected by silver amplification on a chip.

On a single nanostructure level, the linked nanoparticles disaggregate within tens of seconds to minutes depending on the temperature and light intensity applied.

As indicated, in some embodiments, one of the first nucleic acid and second nucleic acid is attached to a surface, wherein the surface is a planar surface, such as can be found on an assay chip. For example, one or more different first nucleic acids (each with at least one probe sequence that can be the same or different) can be attached to the planar surface of an assay chip. The system additionally comprises one or more second nucleic acids (each with at least one target sequence that can be the same or different). In some embodiments, the one or more second nucleic acids can further comprise a detectable moiety, such as any fluorescent or chemiluminescent moiety familiar in the art. The one or more second nucleic acids can be diffused in a liquid media that provides liquid communication with the one or more first nucleic acids on the surface. The system can be employed to ascertain the extent of hybridization between the first and second nucleic acids (or the probe and target sequences therein). The first wavelength of light can be applied to the system to adjust the hybridization stringency between the first and second nucleic acids (or the probe and target sequences therein). Depending on the changes in hybridization states, the presence and/or identity of sequence variations (by way of base-pair mismatches) can be determined.

Accordingly, in another aspect, a method of detecting a sequence variation in a nucleic acid is provided. In one embodiment, the method includes the steps of:

(a) providing a first nucleic acid comprising a photoswitchable molecule and a probe sequence,

wherein the photoswitchable molecule is capable of undergoing a structural change from a first conformation to a second conformation upon illumination by a first wavelength of light;

(b) contacting the first nucleic acid with a second nucleic acid comprising a target sequence that is at least partially complementary to the probe sequence,

wherein the first nucleic acid is contacted with the second nucleic acid under conditions that permit the target sequence to hybridize to the probe sequence, and

wherein the photoswitchable molecule is incorporated into the first nucleic acid at a position four or fewer bases away from the nucleotide position in the probe sequence that hybridizes to the position on the target sequence with a suspected sequence variation;

(c) applying a first wavelength of light at a first photonic energy, thereby promoting a structural change in the photoswitchable molecule that alters a hybridization state of the probe sequence in relation to the target sequence; and

(d) monitoring the hybridization state of the probe sequence in relation to the target sequence,

wherein a conversion to a destabilized, hybridized state or to an unhybridized state between the probe sequence and the target sequence indicates the presence of a sequence variation in the target sequence compared to the probe sequence.

In some embodiments, the method can be performed using the system described hereinabove.

In some embodiments, the first nucleic acid (or probe sequence therein) and the second nucleic acid (or target sequence therein) are partially complementary and partially non-complementary when hybridized. As described above, the partially non-complementary aspect can be a nucleic acid mismatch, an abasic site, a modified base, or result from an insertion, deletion, or translocation event, or a combination of any of the above. In one embodiment, the sequence variation is a single nucleotide polymorphism (SNP). The term SNP refers to a variation occurring at a single nucleotide position in the sequence of nucleic acids (e.g., DNA) shared among members of a group. The group can be a biological designation, such as species or sub-species. The group can also encompass different copies of a particular chromosome. Alternatively, the group can be a batch of nucleic acids produced from the same source, process, or synthesis technique. It will be appreciated that the sequence variation can be a single SNP or multiple SNPs (e.g., potential variations at multiple nucleic acid sites) in the same nucleic acid.

In another embodiment, the sequence variation is an insertion or a deletion that exists in the target sequence of the second nucleic acid relative to the probe sequence in the first nucleic acid. In another embodiment, the sequence variation can be the result of a translocation event reflected in the target sequence of the second nucleic acid relative to the probe sequence in the first nucleic acid. Any of the insertion, deletion, or translocation-based variations described herein can encompass variations of one or more consecutive or nonconsecutive nucleotides. However, it will be appreciated that any sequence variation notwithstanding, sufficient common sequence, manifesting in complementary sequence, must be retained between the probe sequence of the first nucleic acid and the target sequence of the second nucleic acid to permit hybridization thereof.

In a further embodiment, the structural change alters a hybridization state of the probe sequence in relation to the target sequence. In some embodiments, there are at least three hybridization states: 1) a stabilized, hybridized state, 2) an intermediate destabilized, hybridized state, and 3) an unhybridized state. In some embodiments, the structural change destabilizes hybridization of the first nucleic acid (or probe sequence therein) with the second nucleic acid (or probe sequence therein). Thus, the destabilization manifests in converting the hybridization state of the probe sequence in relation to the target sequence from a state of higher stability to a state of lower stability, to an extent even including an unhybridized state with a complete loss of hybridization.

In some embodiments, the method comprises monitoring the hybridization state during and/or after applying the first wavelength of light at the first wavelength. In some embodiments, conversion to a less stabilized state, for example conversion from a stabilized, hybridized state, to an intermediate destabilized, hybridized state or even unhybridized state, can be indicative of a sequence variation (e.g., at least one base-pair mismatch) between the probe sequence and the target sequence. In some embodiments, the promotion of a structural change in the photoswitchable molecule by applying a first wavelength of light converts the hybridization state of the probe sequence in relation to the target sequence from a stabilized, hybridized state to a destabilized, hybridized state. In some embodiments, the presence or identity of a sequence variation is determined by comparing the photonic energy required to cause a detectable change in hybridization state to a control or reference standard value. The reference standard value can reflect the photonic energy required to cause the same conversion of a hybridization state between a probe sequence and its perfect complement (e.g., a “target” sequence with no sequence variation) or a known sequence with greater complementarity than the target sequence under analysis. In some embodiments, the value of the reference standard value is obtained under the same conditions as for the probe and target sequences. For example, in some embodiments, the destabilized hybridization of the probe sequence in relation to the target sequence requires a first amount of photonic energy that is less than a second amount of photonic energy as defined by the amount of photonic energy required to destabilize hybridization of the first nucleic acid with the second nucleic acid to the same extent if they were more complementary. That is to say that the greater the extent of mismatch, the less photonic energy is required to destabilize hybridization. As used herein, the term “photonic energy” refers to the amount of electromagnetic energy absorbed by the photoswitchable molecule. This is sometimes referred to as “photon dose.”

In some embodiments, the promotion of a structural change in the photoswitchable molecule converts the hybridization state of the probe sequence in relation to the target sequence to an unhybridized state. This conversion of hybridization state is indicative of the presence of a sequence variation in the target sequence. Additionally, the conversion occurs under the same conditions wherein a sequence without the sequence variation is not converted to an unhybridized state, including the application of the first wavelength of light at the same photonic energy.

Because photonic energy can be controlled by the application of a specific wavelength at a specific photonic energy and for a specific exposure time, the provided system and methods afford great control over when a photoswitch occurs, and to what extent it occurs. In this regard, if many photoswitchable molecules are incorporated into a nucleic acid, the switching light can be configured to either provide sufficient energy to the system so as to instantly switch all of the photoswitchable molecules, or the switching energy may be delivered more slowly, such that the photoswitchable molecules switch over the course of an elongated timeframe.

It will be appreciated that, depending on the selection of photoswitchable molecules and wavelengths of light, in some embodiments the promotion of a structural change in the photoswitchable molecule by applying a first wavelength of light converts the hybridization state of the probe sequence in relation to the target sequence from a destabilized, hybridized state to a stabilized, hybridized state.

In some embodiments, environmental conditions that can also affect hybridization of probe and target sequences, such as temperature, ionic concentration, and chemical reagent concentration, are held substantially constant during the application of the first wavelength and the monitoring of hybridization state.

Accordingly, the photoswitchability can be used for the detection of base-pair mismatches. The melting temperature of mismatched DNA, for example, is lower than otherwise complementary DNA. This difference is further amplified using the disclosed photoswitchable systems by the cooperative “melting” of DNA during the destabilization from the photoswitchable molecule. For example, the melting temperatures of azoDNA-AuNP with perfect and one base-pair mismatched linkers are ˜60 and ˜37° C., respectively. The rate of photoswitching at 30° C. is therefore expected to be significantly higher for aggregates cross-linked with a mismatched sequence than the perfect complementary DNA. Indeed, after UV irradiation of 30 minutes, the solution of the mismatched linker becomes red due to the SPR of single nanoparticles, while the solution of perfectly matched linker remains the same.

In at least one aspect, the methods and systems disclosed herein enable the detection of a single base-pair mismatch using light as the probe, as mismatched DNA dehybridizes faster at a given temperature than perfectly matched DNA. The photoswitchable plasmonic property is reversible and can be cycled many times to yield light modulated scattering and absorption signals. Because rationally designed DNA probes can be used to detect various types of analytes, such as proteins, ions and small molecules, the modulation in optical signal upon binding of the analyte presents a unique sensing platform with broad applications, especially in standoff detection.

In sensing applications, the invention allows hybridization stringency between perfect and mismatched sequences to be achieved by controlled photon dose, or controlled photon dose in conjunction with conventional thermal or saline wash conditions.

In another embodiment, the photoswitchability of the system depends on temperature and/or ion (e.g., salt) concentration. In one embodiment, a temperature and an ionic concentration of the solution does not change during said step of altering the hybridization property.

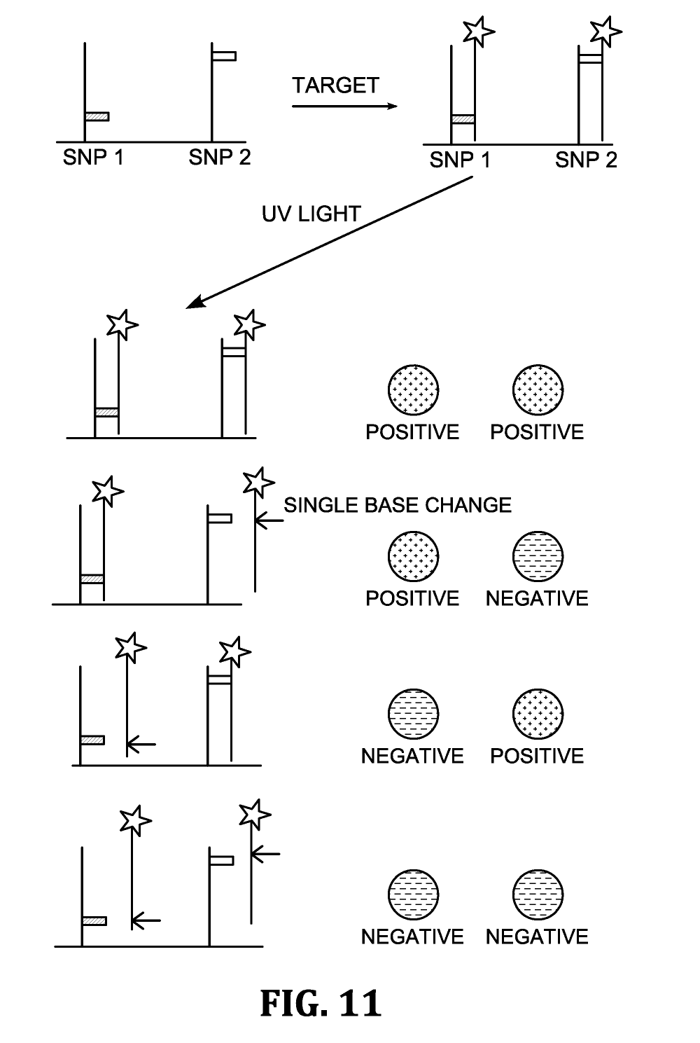

In another embodiment, the photoswitch-modified nucleic acids can be used in chip-based assays. For example, FIGS. 11A and 11B schematically illustrate an array of two distinct probe sequences (each as part of independent “first nucleic acids,” as used herein) that are attached to a planar surface. Each probe contains a photoswitchable molecule (hatched and open rectangles) at a different location, each of which corresponds to a location of distinct suspected SNPs. A sample of fluorescently-tagged nucleic acids containing potential target sequences are introduced and allowed to hybridize to the various probe sequences (FIG. 11). A first wavelength of light is applied at a first photonic energy. The potential results are illustrated in FIG. 11. A loss of hybridization is observed if a base-pair mismatch is present, which indicates the presence of the SNP in the target sequence. If no loss of hybridization is observed, no mismatch can be inferred. It will be appreciated that such assay configuration can be scaled up to contain a large plurality of different probe sequences attached to distinct spots on the planar surface of the assay chip. Accordingly, such a set-up can be used to test for any number of different SNPs, or other known sequence variations, present in a population.

As described below, the inventors determined that different mismatches appearing in local sequences result in different measurable quantum yields when the azobenzene is located within four nucleotide positions. Properly placed photoswitchable molecules in rationally designed probes can provide a unique “fingerprint” by virtue of exhibiting a unique, identifiable quantum energy profile to enable the identification of specific sequence variations. Accordingly, in some embodiments, the method further comprises applying a second photonic energy, which is greater than the first photonic energy, of the first wavelength. In some embodiments, the increased photonic energy leads to further destabilization between the probe sequence and the target sequence. As the photonic energy is increased with the second photonic energy (and thereafter with potential third, fourth, fifth, etc., photonic energies), the hybridization state for the probe/target sequence duplex is monitored. When the hybridization state is converted to a less stable state, the particular photonic energy is noted and can be compared to the photonic energies of known sequence variations. The photonic energy profiles for destabilization of hybridization of known sequences can be provided, for example, in a look up table or can be generated simultaneously as a reference standard assay. With the comparison of photonic energy profiles, the identity of a sequence variation in a target sequence can be determined.

Embodiments of this aspect encompass all types of nucleic acids, as described in more detail above.

Embodiments of this aspect encompass all types of photoswitchable molecules, their various modes of incorporation into the nucleic acids, their configuration changes in response to specific light wavelengths, and their effects on the nucleic acids, as described in more detail above. For example, in one embodiment, the photoswitchable molecule is incorporated into the first nucleic acid sequence. In another embodiment, the photoswitchable molecule is incorporated into the second nucleic acid sequence.

Embodiments of this aspect encompass all types of wavelengths. Persons of skill in the art will be able to select appropriate wavelengths depending on the specific photoswitchable molecule and intended application, as described in more detail above.

In some embodiments, at least one of the first nucleic acid and the second nucleic acid is attached to a surface, as described in more detail above. In one embodiment, the surface is a surface of a core, wherein a core is defined as a particle of micro- or nano-scale size, depending on various factors described below. In other embodiments, the surface is a planar surface, such as can be found on an assay chip.

In another aspect, the disclosure provides a method for designing a probe for detecting sequence variation in a nucleic acid. In one embodiment, the method comprises:

(a) obtaining the sequence of a reference nucleic acid, or a complement thereof;

(b) determining the location in the reference nucleic acid sequence, or the complement thereof, of a suspected sequence variation; and

(c) designating in the reference nucleic acid sequence, or the complement thereof, at least one position within four nucleic acid positions of the location of the suspected sequence variation to receive the incorporation of a photoswitchable molecule, wherein the photoswitchable molecule is capable of undergoing a structural change from a first conformation to a second conformation upon illumination by a first wavelength of light.

The reference sequence can be a wild-type sequence or any reference to which other sequences are compared for variation. Specific locations within the reference sequence where sequence variation is suspected to occur in the population or group of sequences are noted. Considering the discovery of the present inventors, described in more detail below, a photoswitchable molecule, such as azobenzene, is designated for placement into the reference sequence at a location of four or fewer bases from the location of the suspected sequence variation. It will be appreciated that consideration should be given to the character of the source population or group of target sequences that are available for assaying. For example, if the source population or group of target sequences only contains single stranded nucleic acids (e.g., positive or negative strands only), then the probe comprising a reference sequence should contain a reference sequence that is the complement to the positive (or negative) strand available in the source population or group of target sequences. Accordingly, hybridization can occur between the probe and the target sequence in the assay for an informative result. If the source population or group of target sequences contains both positive and negative strands (e.g., is double stranded), then the probe can contain the reference sequence in either the positive or negative strand.

In some embodiments, a nucleic acid is synthesized or otherwise produced that contains a reference sequence and a photoswitchable molecule at a position within four nucleobases of the suspected sequence variation. Exemplary methods for producing such probes are described in more detail below.

The nucleic acids, photoswitchable molecules, wavelengths of light, and other elements of this aspect, are described in more detail above.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

Disclosed are materials, compositions, and components that can be used for, can be used in conjunction with, can be used in preparation for, or are products of the disclosed methods, compositions, and systems. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material, such as those described elsewhere herein or as known in the art.

Publications cited herein and the subject matter for which they are cited are hereby specifically incorporated by reference in their entireties.

Characterization of Sequence Variations Using Quantum Yield

The following is a description of an exemplary approach for the development of using photoisomerization quantum yield of azobenzene-modified DNA to characterize local sequence variations. It is noted that the following description is included for the purpose of illustrating, not limiting, the described embodiments.

### INTRODUCTION

Molecular photoswitches such as azobenzene have a long history of application in fields ranging from material science to biology. Recently, the modification of DNA with these molecules has also allowed the addition of stimulus-response functionality to a wide range of DNA-based technologies.

Notably, an azobenzene-modified phosphoramidite has been developed, which allows virtually any DNA sequences amenable to solid-phase synthesis to be readily functionalized with multiple azobenzene photoswitches. In this approach, the azobenzenes are incorporated by tethering them to additional sugar/phosphate linkages along the DNA backbone via a d-threoninol group (FIG. 1B). Despite some structural distortion of the double helix resulting from the volume of the extra phosphate and azobenzene moieties, the incorporation of a trans form of azobenzene stabilizes a DNA duplex by intercalation between the neighboring bases. This stabilization raises the melting temperature of the azobenzene-modified DNA above that of an otherwise identical native sequence. Absorption of UV light (320-370 nm) excites the S₀-S₂ transition of the azobenzene groups, promoting trans-to-cis photoisomerization (see FIG. 6). In the cis form the azobenzenes destabilize the DNA duplex, significantly lowering the melting temperature of the DNA. Blue light (˜450 nm) converts the cis form back to trans, thereby permitting reversible optical control of DNA hybridization.

As described herein, these characteristics are harnessed to develop DNA-hybridization assays capable of differentiating single-base mismatches using a photonic stringency wash.

Such an application is sensitive to the quantum yield for azobenzene photoisomerization: the total amount of optical energy required to achieve DNA denaturation. Surprisingly, while the quantum yield for azobenzene photoisomerization is known to depend on the local environment, there is very little data on how the quantum yield for photoisomerization of azobenzene is affected by incorporation into different DNA sequences.

Here, we address this question by studying the quantum yield of trans-to-cis photoisomerization of azobenzenes inserted into DNA sequences via the popular Asanuma chemistry. We show that the quantum yield for photoisomerization decreases upon incorporation into single stranded DNA (ssDNA), and decreases further upon incorporation into double stranded DNA (dsDNA). Importantly, we show that the quantum yield in dsDNA is sensitive to the melting temperature of the sequence and very sensitive to the presence of local mismatches. These data indicate that sequence variations, e.g., base-pair mismatches between complement strands, can be detected and potentially identified using photoisomerization of azobenzene-modified DNA

Results and Discussion

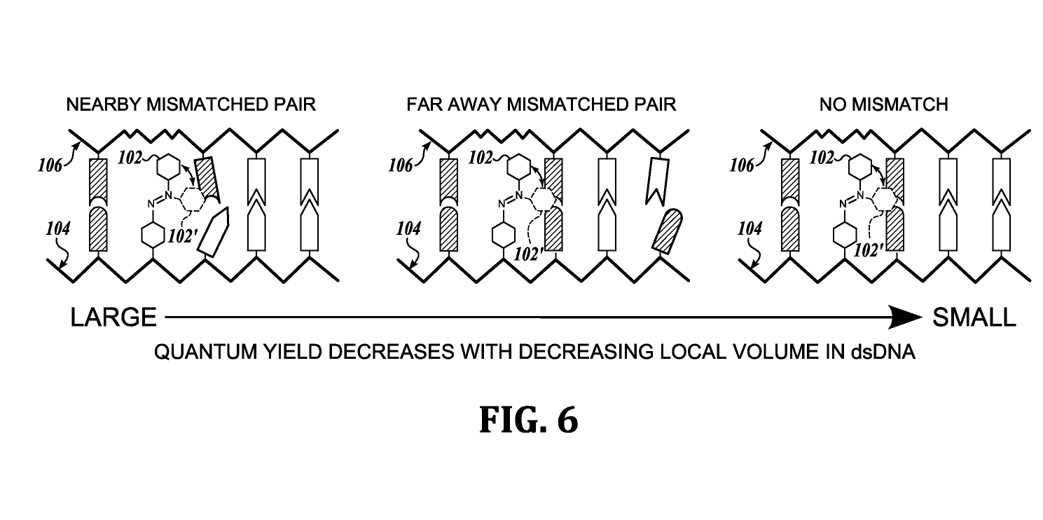

To assess how incorporation into various DNA sequences alters the trans-to-cis photoisomerization of azobenzene we measured the quantum yields in various modified DNA molecules by quantifying the fraction of cis-azobenzene as a function of UV (330 nm) irradiation time (FIG. 7) using UV-vis absorption spectroscopy. FIG. 1A compares the absorption spectra of free trans-azobenzene dissolved in isooctane and trans-azobenzene incorporated in ssDNA in phosphate buffer. The free azobenzene exhibits the typical π to π* absorption at 315 nm, whereas the azobenzene incorporated into ssDNA has its absorption band shifted to about 340 nm.

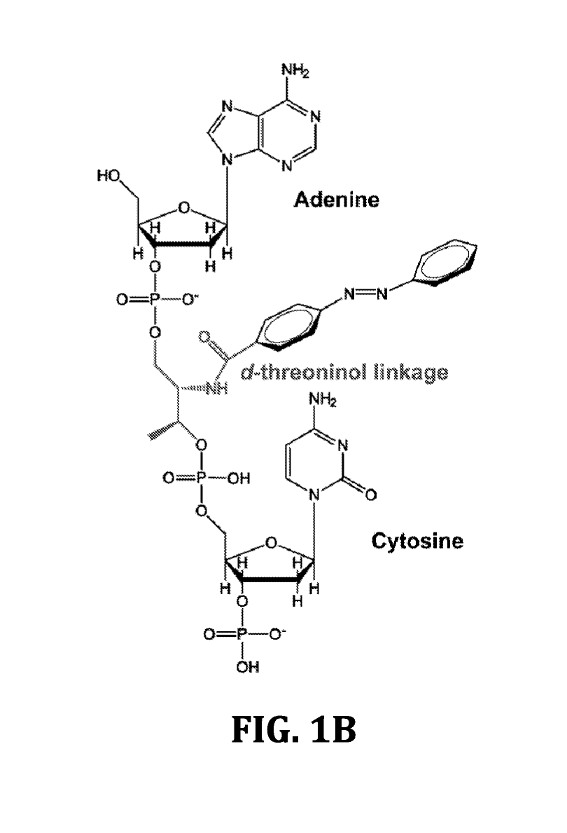

FIG. 2A compares the fraction of cis-azobenzene as a function of the integrated photokinetic factor (excitation time converted to photon dose to account for the changing absorption of the solution over time) at 28° C. for free azobenzene, azobenzene attached via d-threoninol linkages to a ssDNA sequence (SEQ ID NO:1) and the same DNA sequence hybridized to its (azobenzene-free) complement (SEQ ID NO:2). For free azobenzene, the fraction of cis-azobenzene at the photo-stationary state is 0.93, which decreases to 0.51 for azobenzene incorporated into ssDNA, and 0.15 for azobenzene incorporated into dsDNA. We extracted the photoisomerization quantum yield from the curves in FIG. 2A by fitting the data to Equation 1:

y=(y₀−y_{∞})exp(−Ax)+y_{∞} Equation 1

where x is the integrated photokinetic factor defined via Equation 2, y is the fraction of cis-azobenzene (y₀ and y_{∞} are the fractions of cis-azobenzene before photoisomerization and at the photo-stationary state), and A is a pre-factor related to the trans-to-cis quantum yield (see supporting information for details). The integrated photokinetic factor is given by:

$$x ( t ) = {\int }_{0}^{t} \frac{1 - {10}^{- ab s ( t )}}{abs ( t )} d t \qquad Equation 2$$

FIG. 2A shows fits of Equation 1 to the data as solid lines. From these fits we obtained a quantum yield for free azobenzene of 0.094±0.004 when excited at 330 nm.

As can be seen from the decrease in cis-azobenzene fraction in the photo-stationary state at a given illumination intensity, incorporation into the ssDNA sequence decreases the photoisomerization quantum yield by a factor of ˜3 to 0.036±0.002. Hybridization of the ssDNA to its complement (Seq-complement) results in an even more dramatic decrease of the quantum yield to 0.0056±0.0008. Since the DNA solutions do not absorb at the UV excitation wavelength used here, we attribute these differences entirely to the attachment of the azobenzene to the DNA sequences.

While the structure—ssDNA and dsDNA—has a strong influence on the azobenzene photoisomerization quantum yield, we also find that the sequence of the dsDNA—particularly mismatches in the sequence near the azobenzene site—plays a role. To explore sequence effects, we used azobenzene-modified dsDNA with one sequence having azobenzene incorporated in the center, and the other sequence bearing a single-base mismatch at a varying distance from the azobenzene site (see Table 1 for sequence data). FIG. 2B compares the fraction of cis-azobenzene as a function of the integrated photokinetic factor (Equation 2) for several different dsDNA sequences at 28° C. By fitting Equation 1 to the data in FIG. 2B, we extracted the photoisomerization quantum yields of the azobenzene in these different dsDNA sequences. The quantum yields for all studied sequences are summarized in Table 1. FIG. 2B and the accompanying fits show that the varying fractions of cis-azobenzene achieved at the photo-stationary state are due to sequence-dependent variations in the azobenzene photoisomerization quantum yield.

TABLE 1
Quantum yields and melting temperatures (Tₘ) of the
azobenzene-modified DNA

| | | SEQ ID | | Tₘ |
|---|---|---|---|---|
| Names | Sequences | NO: | Quantum Yield | (° C.) |
| ssDNA | 5′-AGACTGAACXCAATGTATG-3′ | 1 | 0.036 ± 0.002 | |
| | X: azobenzene | | | |
| Seq-mm- | 5′-AGACTGAACX [symbol] AATGTATG-3′ | 1 | 0.020 ± 0.001 | 46.7 |
| abasic | TCTGACTTG [symbol] TTACATAC | 3 | | |
| (mm: | [symbol] : abasic site | | | |
| mismatch) | | | | |
| Seq-mm1T | 5′-AGACTGAACX [symbol] AATGTATG-3′ | 1 | 0.016 ± 0.001 | 48.0 |
| | TCTGACTTG [symbol] TTACATAC | 4 | | |
| Seq-mm1C | 5′-AGACTGAACX [symbol] AATGTATG-3′ | 1 | 0.015 ± 0.001 | 48.0 |
| | TCTGACTTG [symbol] TTACATAC | 5 | | |
| Seq-mm1A | 5′-AGACTGAACX [symbol] AATGTATG-3′ | 1 | 0.0078 ± 0.0007 | 48.0 |
| | TCTGACTTG [symbol] TTACATAC | 6 | | |
| Seq-mm2 | 5′-AGACTGAACXC [symbol] ATGTATG-3′ | 1 | 0.011 ± 0.001 | 52.0 |
| | TCTGACTTG G [symbol] TACATAC | 7 | | |
| Seq-mm3 | 5′-AGACTGAACXCA [symbol] TGTATG-3′ | 1 | 0.0070 ± 0.0002 | 54.0 |
| | TCTGACTTG GT [symbol] ACATAC | 8 | | |
| Seq-mm4 | 5′-AGACTGAACXCAA [symbol] GTATG-3′ | 1 | 0.0069 ± 0.0006 | 54.0 |
| | TCTGACTTG GTT [symbol] CATAC | 9 | | |
| Seq- | 5′-AGACTGAACXCAATGTATG-3′ | 1 | 0.0056 ± 0.0008 | 60.0 |
| complement | TCTGACTTG GTTACATAC | 2 | | |

Of the sequences used in FIG. 2B, the lowest quantum yield of 0.0056±0.0008 comes from azobenzene-modified dsDNA with no mismatches (Seq-complement). The quantum yield increases to 0.011±0.001 for dsDNA having an A·C mismatch two bases away from the azobenzene position (Seq-mm2). In contrast, a C·T mismatch immediately next to the incorporated azobenzene (Seq-mm1T) increases the quantum yield by an additional 45% to 0.016±0.001. Finally, we measure the largest quantum yield (0.020±0.001) using modified dsDNA having an abasic site (with no purine or pyrimidine, FIG. 1C, Seq-mm-abasic) as the nearest neighbor to the azobenzene in the unmodified sequence.

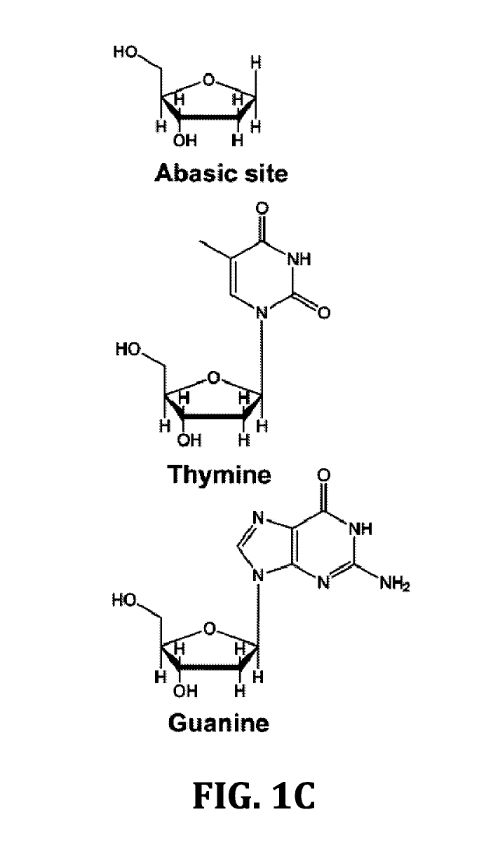

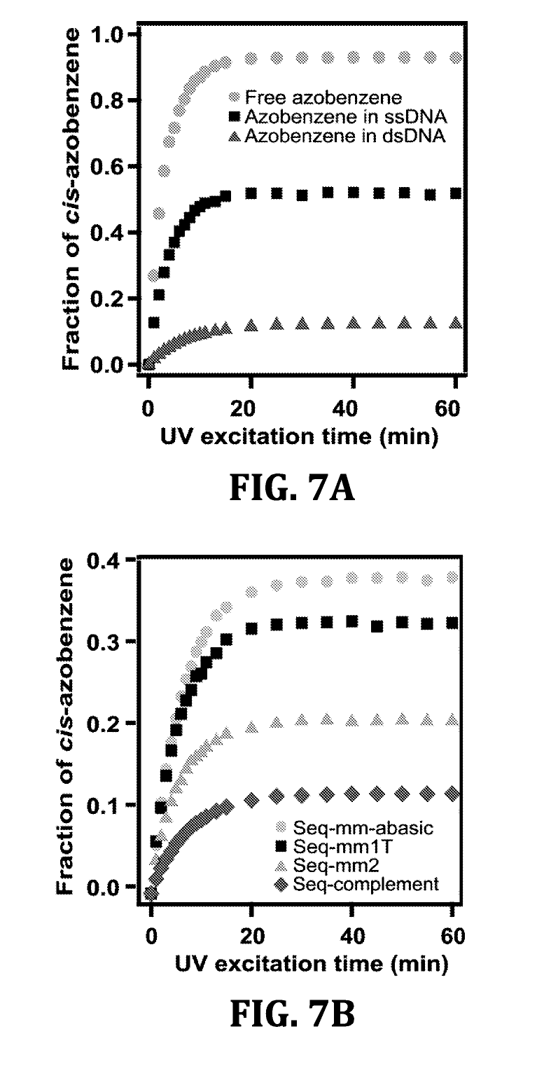

To examine the effects of dsDNA stability and DNA sequence on quantum yield, FIG. 3 plots the quantum yield as a function of the measured melting temperature (Tₘ) (FIG. 8) of the modified dsDNA sequences listed in Table 1. Broadly, the data show that the azobenzene quantum yield tends to decrease with increasing Tₘ of the embedding DNA sequence. We measure the highest azobenzene quantum yield (0.020±0.001) for the dsDNA with the lowest Tₘ (46.7° C.) (Seq-mm-abasic). Likewise, we measure the lowest photoisomerization quantum yield (0.0056±0.0008) for the dsDNA with the highest Tₘ (60.0° C.) (Seq-complement). These data confirm that photoisomerization can be used to modulate Tₘ, but importantly, our work shows that the converse is also true: the Tₘ of the dsDNA sequence can, in turn, affect trans-to-cis isomerization efficiency.

Looking at these endpoints, one might be tempted to conclude that Tₘ is the primary controlling factor in determining the azobenzene quantum yield. However, closer inspection of the data reveals that more complicated effects are at work. For instance, FIG. 3 shows that three sequences with very similar Tₘ have dramatically different quantum yields for azobenzene photoisomerization. Interestingly, all three of these sequences have a single-base mismatch immediately next to the position of the azobenzene (Seq-mm1T, Seq-mm1C, Seq-mm1A). The photoisomerization quantum yield is less sensitive to mismatches that are two or more bases away from the position of the azobenzene (e.g., Seq-mm2, Seq-mm3, Seq-mm4).

We propose that the variations we observe in photoisomerization quantum yield when the azobenzene is incorporated into DNA are largely due to differences in the local free volume available to the azobenzene in the different sequences. Generally, the azobenzene quantum yield is known to depend on the free volume surrounding the azobenzene site, and this explanation would be consistent with our results that the quantum yield decreases on going from an azobenzene free in solution to being incorporated in ssDNA to being incorporated in dsDNA. Furthermore, this hypothesis could account for the large differences we observe between single-base mismatches that are next to the azobenzene site, and those that are further removed: distortions in the double helix are known to recover over a decay length of only a few bases.

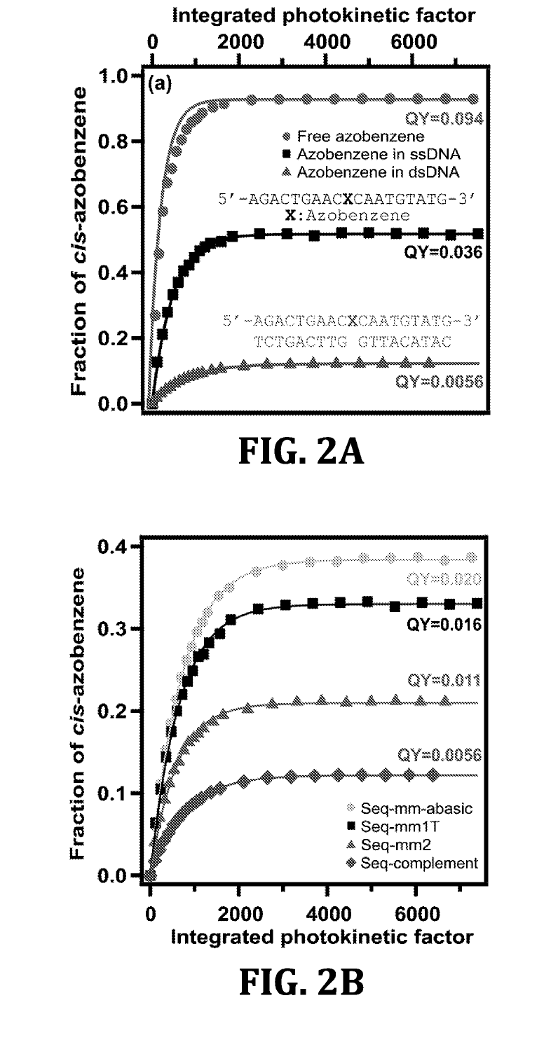

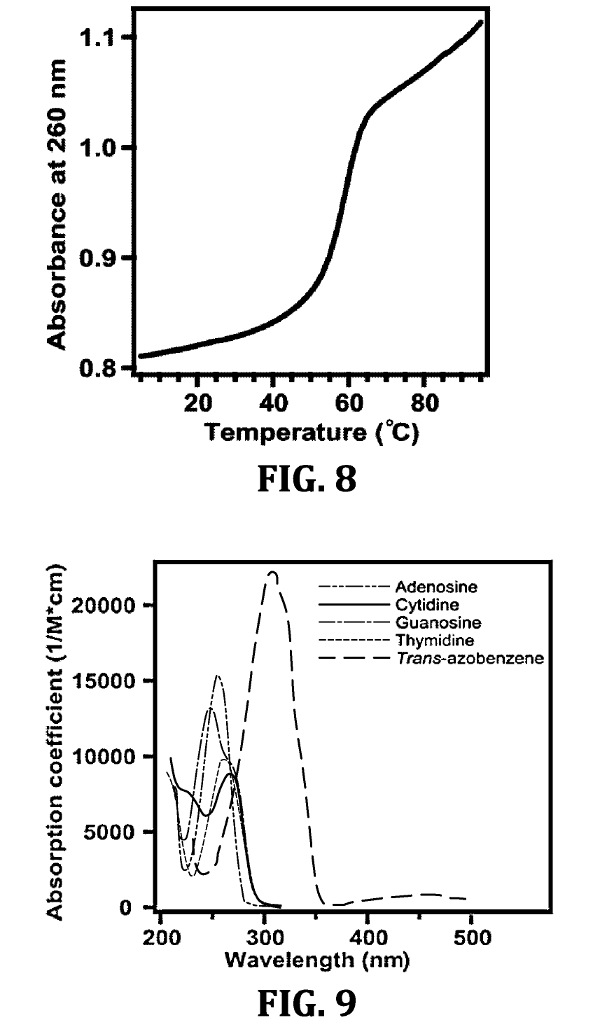

However, it is also possible that electronic interactions, including both energy and charge transfer between the azobenzene and the nucleic acid bases are modulating the quantum yield by changing the energy relaxation pathways available to the azobenzene on an ultrafast timescale. In this case, one would explain the sequence-dependent differences in quantum yield as arising from different electronic interactions between the azobenzene and the different bases. To test this alternative hypothesis, FIG. 4 plots the photoisomerization quantum yield (bars) for the azobenzene incorporated in the middle of 18-base ssDNAs comprising polydeoxyadenosine (poly(dA)), polydeoxycytidine (poly(dC)) and polydeoxythymidine (poly(dT)). In comparison, FIG. 4 also plots the oxidation potential of DNA nucleotides (dots) measured in buffer (pH=7). We note that if electronic coupling were dominant, one might expect to see some correlation between oxidation potential of the neighboring bases and the quantum yield. However, FIG. 4 shows that the quantum yield of azobenzene contained in poly(dA), poly(dC), and poly(dT) sequences appears uncorrelated with the oxidation potentials of the bases. On the other hand, it is known that poly(dT) is more flexible than poly(dA). This result also fits well with the free volume hypothesis: azobenzene in more structurally flexible poly(dT) has a higher quantum yield than azobenzene in more rigid poly(dA). Likewise, if energy transfer were dominant, one might expect a correlation between the positions of the UV-vis spectra of the nucleotides and the measured quantum yields—however we were unable to observe such a correlation (FIG. 9). Thus, we propose that free volume is likely to be more important, while acknowledging that it will be difficult to completely separate electronic and structural control over variations in the azobenzene photoisomerization quantum yield because an increase in local free volume around the azobenzene should also tend to weaken intermolecular electronic interactions.

Finally, we can use the observed variation of azobenzene quantum yield with the DNA sequence to explain the properties of azobenzene-modified DNA that facilitate its use in novel DNA-hybridization assays. Previously we have shown that using only optical inputs, DNA sequences containing single-base mismatches can be resolved in hybridization experiments involving gold nanoparticles that are heavily functionalized with azobenzene-modified DNA. The resulting photonic hybridization stringency wash works because the denaturation of azobenzene-modified DNA strands occurs at lower photon doses for sequences with less complementarity. The results presented herein provide a fundamental mechanistic understanding of this process: partially mismatched sequences denature at lower photon doses because the azobenzenes in those sequences photoisomerize more efficiently than azobenzenes in perfectly complementary sequences.

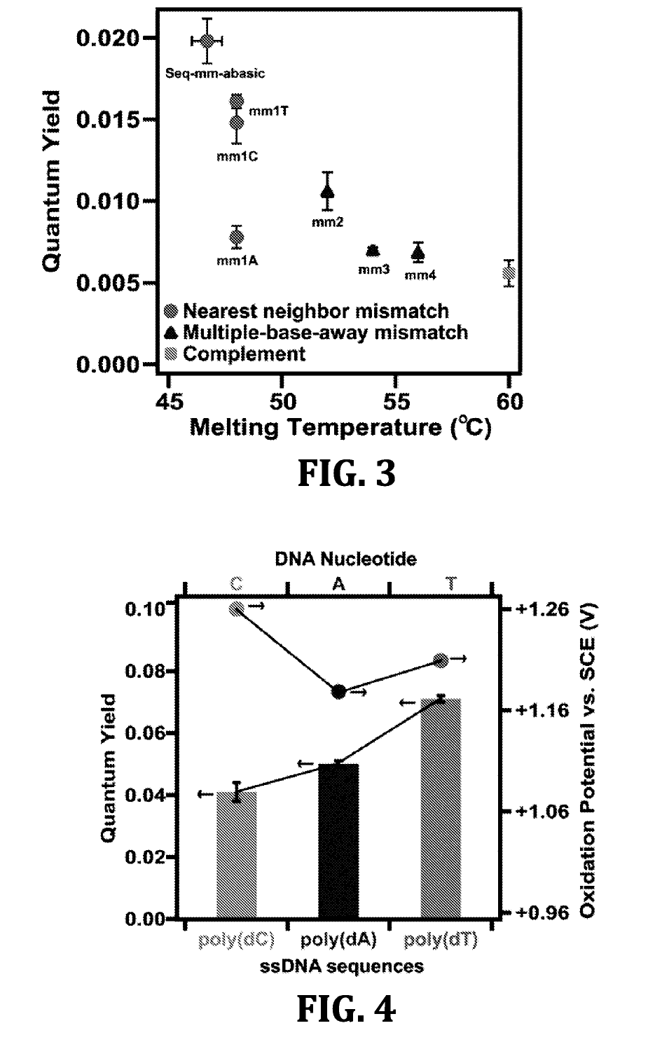

To test this hypothesis, we measured the trans-to-cis isomerization quantum yield of azobenzenes incorporated in dsDNAs in a classic three-strand capture assay using a linker strand as shown in FIG. 5A. The assay consists of the capture DNA modified with multiple azobenzenes (“Sequence-1,” set forth in SEQ ID NO:10), the unmodified probe DNA (“Sequence-2,” set forth in SEQ ID NO:11) and the unmodified target/linker (“Sequence-3,” set forth in SEQ ID NOS:12-15) that cross-hybridizes with the capture and probe sequences. We varied the target sequence by introducing a single base mismatch in the center where it will form a mismatched base pair next to the azobenzene (see sequences of “Sequence-3” in FIG. 5A, SEQ ID NOS:12-15). We measure the lowest quantum yield for the perfectly complementary sequence (“Seq-perfect,” SEQ ID NO:12), and observe an increase in quantum yield from Seq-mmA (SEQ ID NO:14), to Seq-mmC (SEQ ID NO:15) and to Seq-mmT (SEQ ID NO:13)—all having the single-base mismatch next to one of the azobenzenes. In order to compare the trend in quantum yield with that of optical hybridization stringency, we functionalized gold nanoparticles with the same capture and probe DNAs and measured the disaggregation rate of nanoparticle conjugates cross-linked by the same target DNA, which we have previously shown can exhibit photon-dose-controlled hybridization stringency. We quantify the disaggregation rate by monitoring the localized surface plasmon resonance (LSPR) peak shift of nanoparticle conjugates, which is expected a blue shift as conjugates undergo photoinduced dissociation. Indeed, the inset in FIG. 5B shows that the photon-dose dependence of the nanoparticle disaggregation process follows the exact same sequence-dependent order as the measured azobenzene quantum yields, providing strong evidence that the two trends are linked.

### CONCLUSION

We have shown that the trans-to-cis photoisomerization quantum yield for azobenzene decreases upon incorporation into DNA, and is sensitive to both the local DNA sequence and DNA hybridization state. In general, the photoisomerization quantum yield tends to increase as the Tₘ of the attached dsDNA decreases. However, the biggest variations in quantum yield are associated with dsDNAs bearing a single-base mismatch immediately next to the azobenzene site. We propose that these variations arise due to the structural fluctuations caused by the adjacent mismatched base inducing an increase in the local free volume. These results provide a mechanism to explain optically-controlled DNA hybridization stringency and permit the detection and characterization of base-pair mismatches between partially complementary strands.

Experimental Methods

Materials.

Unmodified DNAs and azobenzene-modified DNAs were purchased from Integrated DNA Technology (IDT Inc, IA). All sequences used are shown in Table 1 and FIG. 5(a). Water was deionized to 18.0 MOhm with the Millipore filtration system.

Preparation of dsDNA Solution.

Aliquots of lyophilized DNA were dissolved in water and a desired amount of DNA aqueous solution was then brought to 0.01 M phosphate buffer (pH=6.5), 0.1 M NaCl and 0.02% sodium azide. Equal moles of complementary DNAs were mixed at room temperature and annealed at 95° C. for 5 min. The concentration of azobenzene modified dsDNA for quantum yield tests was prepared to be 10 μM; and the concentration for Tₘ test was 2 μM. The dsDNA solution was kept at 4° C. before use.

Preparation of the dsDNA that has Three-Sequence Structure.

The sequences for the three-strand capture assay are shown in FIG. 5A. Sequence-1 and Sequence-2 are both complementary to part of Sequence-3 (in each of its four variations). The annealing process consisted of two steps. First, Sequence-2 and Sequence-3 were combined and annealed at 95° C. for 5 min, followed by gradual cooling to 55° C. and being held at 55° C. for 30 min. Then, Sequence-1 was added to the solution and annealed for an additional 10 min at 55° C. The DNA solution was lowered to room temperature and kept at 4° C. before use.

Quantum Yield Measurements.

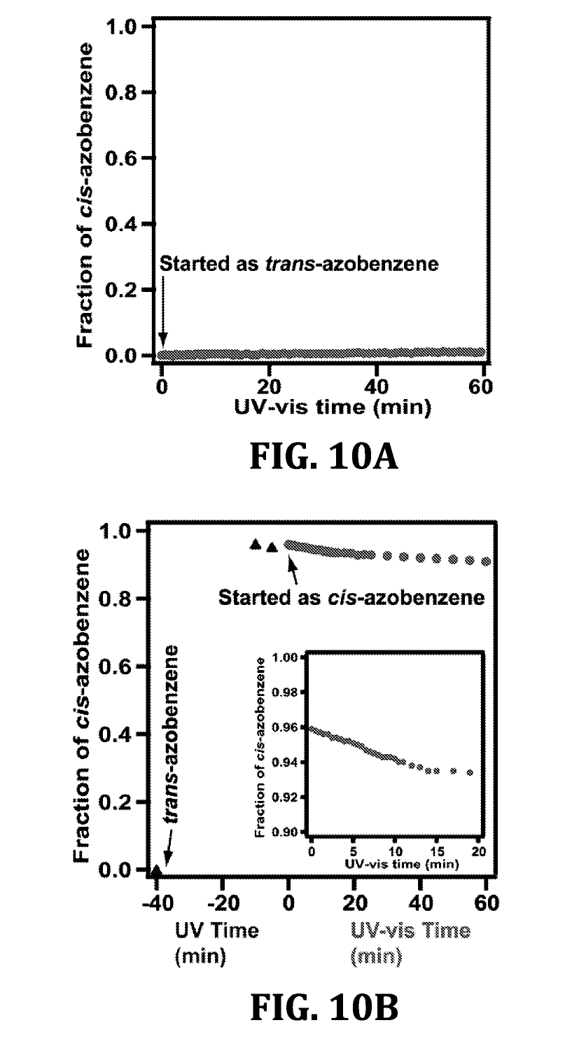

The UV irradiation setup for quantum yield measurement consisted of an LED light source centered at 330 nm with FWHM less than 10 nm (UVTOP325HS Sensor Electronic Technology, Inc.), a home-made aluminum stage, a quartz cuvette with 1 cm optical path length, a stir plate, and a temperature controller. The temperature of the DNA solution as a function of temperature controller set point (measured in the aluminum stage) was calibrated using a thermometer. The temperature of the DNA solution was kept at 28° C. for all quantum yield measurements using the calibrated temperature controller. The UV LEDs were warmed up for 1 h and then the illumination intensity was monitored using a silicon photodiode positioned at the other end of the aluminum stage. The UV intensity was typically 0.37 to 0.42 mW/cm². Azobenzene-modified DNA solutions were added to the quartz cuvette and were thermally equilibrated at 28° C. for 1 h in the dark before UV irradiation. During UV irradiation, UV-vis absorption spectra were recorded by an Agilent 8453 UV-vis spectrometer every 1 min for 15 min and then every 5 min for the rest 45 min. The DNA solution was stirred during the measurement. We verified that the low intensity white light source of the spectrometer had no significant effect on the photoisomerization process (FIGS. 10A and 10B).

Melting Temperature Measurements.

An Agilent 8453 UV-vis spectrometer operating in the thermal denaturation mode was used to measure the melting temperature. The temperature ramp started at 5° C. and ended at 95° C. with 2° C. step interval and a 5 min hold time. Melting temperature was determined as the temperature at which the first derivative of the absorbance vs. temperature plot was maximum. See selected data in FIG. 8.

Quantum Yield Calculations.

The azobenzene trans-to-cis isomerization quantum yield is the ratio between the number of isomerized trans-azobenzenes and that of absorbed photons at the actual time (a differential quantum yield). We use the following isomerization rate equation:

$$\frac{d {[ cis ]}_{t}}{d t} = \frac{I * l * ( 1 - {10}^{- abs ( t )} ) * {φ}_{trans} * {ɛ}_{trans}}{V * abs ( t )} ( {[ trans ]}_{0} - {[ cis ]}_{t} ) - \frac{I * l * ( 1 - {10}^{- abs ( t )} ) * {φ}_{cis} * {ɛ}_{cis} }{V * abs ( t )} [ cis ] t \qquad Equation S1$$

where [cis]ₜ is the concentration of cis-azobenzene at time t; [trans]₀ is the concentration of trans-azobenzene before photoisomerization, which we assume to be the total concentration of azobenzene; I is the intensity of the excitation; l is the beam path length of the UV-vis absorption measurement; abs (t) is the absorbance of the sample at the excitation wavelength, and t is time; φₜᵣₐₙₛ and φ_{cis} are the quantum yields of trans-to-cis and cis-to-trans isomerization; εₜᵣₐₙₛ and ε_{cis} are the absorption coefficients at the excitation wavelength of trans-azobenzene and cis-azobenzene; V is the volume. At the photo-stationary state,

$$\frac{d {[ cis ]}_{t}}{d t} = 0 ,$$

we get the following relation:

$$\frac{{φ}_{trans}}{{φ}_{cis}} = \frac{{[ cis ]}_{∞} * {ɛ}_{cis}}{( {[ trans ]}_{0} - {[ cis ]}_{∞} ) * {ɛ}_{trans}} \qquad Equation S2$$

where [cis]_{∞} is the concentration of cis-azobenzene at photo-stationary state. By defining the fraction of cis-azobenzene as

$$y = \frac{[ cis ]}{{[ trans ]}_{0}}$$

and the fraction of trans-azobenzene as 1−y, and by solving Equations S1 and S2, we obtain the following equation

$$ln \frac{{y}_{∞} - y}{{y}_{∞} - {y}_{0}} = - \frac{l * l * {φ}_{trans} * {ɛ}_{trans}}{V * {y}_{∞}} {\int }_{{t}_{0}}^{t} \frac{1 - {10}^{- {abs}^{λ} ( t )}}{{abs}^{λ} ( t )} d t \qquad Equation S3$$

By replacing

$$\frac{I * l * {φ}_{trans} * {ɛ}_{trans}}{V * {y}_{∞}}$$

as pre-factor A, and

$${\int }_{{t}_{0}}^{t} \frac{1 - {10}^{- {abs}^{λ} ( t )}}{{abs}^{λ} ( t )} d t$$

as variable x, we obtain Equation 1, shown above.

Generating a Chip-Based Assay

The following is a description of an exemplary approach for generating a chip-based assay to detect sequence variations in one or more nucleic acids using azobenzene-modified probes for each target nucleic acid of interest.

### INTRODUCTION

As described above, it has been discovered that synthetic azobenzene-modified DNA enables the use of light to control DNA hybridization stringency with a resolution that can differentiate even a single base change in the complementary sequence. This optical-controlled selective phenomenon is due to the fact that light induces the isomerization reaction of trans-azobenzene to the cis-azobenzene, which destabilizes the hybridization of the complementary strands. The inventors further uncovered that azobenzene's photo-induced isomerization depends on DNA sequences and relative location of mismatched base pairs.

Methods

Utilizing the sequence-dependent mechanism, a design for a DNA chip that can simultaneously detect multiple single polymorphism nucleotides located in proximity is proposed.

Multiple azobenzene-modified probes are rationally designed with azobenzenes at varied positions to collect differential signals of multiple SNPs that are located at different sites in the same gene. It will be appreciated, however, that the distinct probes can also be configured to contain probes corresponding to different genes. FIG. 11 depicts the design in a simplest fashion. Specifically, the embodiment illustrated in FIG. 11 shows two distinct probes for simplicity. However, it will be appreciated that such a format can be scaled up to contain hundreds or thousands of specific probes to test for different sequence variations (e.g., SNPs, etc.). Nucleic acid probes bear one azobenzene at the lower region (hatched horizontal bar) which targets for SNP 1 on gene X, while the other azobenzene which targets for SNP 2 (open horizontal bar) is located at the upper region of the probe. After exposing target gene X to the probes in buffer at the optimal temperature, hybridization will occur between the target sequences and the probes. A subsequent 5 min UV irradiation (˜10 mW) is applied the system to develop differential fluorescent patterns that indicate the SNP type (e.g., wild type vs. mutant), as shown in FIG. 11.

The first possible outcome illustrated in FIG. 11 is a “positive, positive” result which corresponds to gene “X” being perfectly complementary to the probes. This means that SNP 1 and SNP 2 are both wild type. The second possible outcome is a “negative, positive” pattern which indicates that gene “X” has a single base change at SNP 1 site, which corresponds to the lower azobenzene region. As described above, the inventors have discovered that when azobenzene is in close proximity to a mismatched base pair (e.g., within about four nucleobase positions), its isomerization quantum yield increases, favoring the dissociation of the target-probe pair. The single base change of SNP1 leads to a separation of gene X from the probe, which results in a less intense fluorescent signal. Hence, a “negative, positive” outcome suggests that SNP 1 is a mutant type and SNP 2 is a wild type, while a similar “positive, negative” outcome suggests that SNP1 is a wild type and SNP 2 is a mutant type. The last outcome is “negative, negative” meaning that SNP 1 and SNP 2 are both mutants.

In conclusion, light-controlled parallel detection of two SNPs is realized due to the sensitive isomerization of azobenzene. The inventors envision that it is possible to run detection of multiple SNPs of many genes on a micro-sized chip by simply turning on the light.

While illustrative embodiments have been illustrated and described, it will be appreciated that various changes can be made therein without departing from the spirit and scope of the invention.

1. A system, comprising:
(a) a first nucleic acid comprising a photoswitchable molecule and a probe sequence, wherein the photoswitchable molecule is capable of undergoing a structural change from a first conformation to a second conformation upon illumination by a first wavelength of light at a first photonic energy, wherein the structural change alters a hybridization property of the first nucleic acid sequence in relation to a target sequence;
(b) a second nucleic acid comprising the target sequence;
wherein the target sequence is partially complementary to the probe sequence, such that the target sequence is configured to hybridize with the probe sequence; and
wherein there is a base-pair mismatch between the target sequence and the probe sequence at a position four or fewer bases away from the photoswitchable molecule; and
(c) liquid media providing liquid communication between the first nucleic acid and the second nucleic acid.

2. The system of claim 1, wherein the first nucleic acid and the second nucleic acid are independently a deoxyribonucleic acid (DNA), a ribonucleic acid (RNA), or a synthetic variant thereof.

3. The system of claim 1, wherein the photoswitchable molecule is an azobenzene, a stilbene, a spiropyran, a fulgide, a diarylethene, a diphenylpolyene, a dihydro-indolizine, a diarylethane, a chromene, a napthopyran, a spiropyran, a fulgide, a fulgimide, a spiroxazine, or any photoswitchable analog thereof.

4. The system of claim 1, wherein the photoswitchable molecule is covalently attached to the first nucleic acid sequence and intercalates between the probe sequence and the target sequence when hybridized.

5-8. (canceled)

9. The system of claim 1, wherein the photoswitchable molecule is azobenzene, and wherein the first wavelength of light is between about 280 nm and 380 nm.

10. The system of claim 1, wherein at least one of the first nucleic acid and the second nucleic acid is attached to a surface.

11. The system of claim 10, wherein the surface is a particle core that is optically detectable by changes in absorption, light scattering, or photoluminescence that are triggered by changes in the hybridization state of the first nucleic acid sequence in relation to the second nucleic acid sequence.

12. (canceled)

13. The system of claim 11, wherein the core has a surface plasmon resonance.

14. The system of claim 10, wherein the surface is a planar surface on a substrate.

15. (canceled)

16. A method of detecting a sequence variation in a nucleic acid, comprising:
(a) providing a first nucleic acid comprising a photoswitchable molecule and a probe sequence;
wherein the photoswitchable molecule is capable of undergoing a structural change from a first conformation to a second conformation upon illumination by a first wavelength of light;
(b) contacting the first nucleic acid with a second nucleic acid comprising a target sequence that is at least partially complementary to the probe sequence;
wherein the first nucleic acid is contacted with the second nucleic acid under conditions that permit the target sequence to hybridize to the probe sequence, and
wherein the photoswitchable molecule is incorporated into the first nucleic acid at a position four or fewer bases away from the nucleotide position in the probe sequence that hybridizes to the position on the target sequence with a suspected sequence variation;
(c) applying a first wavelength of light at a first photonic energy, thereby promoting a structural change in the photoswitchable molecule that alters a hybridization state of the probe sequence in relation to the target sequence; and
(d) monitoring the hybridization state of the probe sequence in relation to the target sequence,
wherein a conversion to a destabilized, hybridized state or to an unhybridized state between the probe sequence and the target sequence indicates the presence of a sequence variation in the target sequence compared to the probe sequence.

17. The method of claim 16, wherein the sequence variation is a single nucleotide polymorphism (SNP).

18. The method of claim 16, wherein the promotion of a structural change in the photoswitchable molecule by applying a first wavelength of light in step (c) converts the hybridization state of the probe sequence in relation to the target sequence from a stabilized, hybridized state to a destabilized, hybridized state, wherein the conversion requires a first amount of photonic energy that is less than a second amount of photonic energy as defined by the amount of photonic energy required to convert the hybridization state of the probe sequence in relation to the target sequence to if the target sequence did not have a sequence variation.

19. (canceled)

20. The method of claim 16, wherein the promotion of a structural change in the photoswitchable molecule by applying a first wavelength of light in step (c) converts the hybridization state of the probe sequence in relation to the target sequence to an unhybridized state, thereby indicating the presence of a sequence variation in the target sequence, and wherein the promotion occurs under conditions wherein a sequence without the sequence variation is not converted to an unhybridized state.

21-22. (canceled)

23. The method of claim 16, wherein the first and second nucleic acids are independently a deoxyribonucleic acid (DNA), a ribonucleic acid (RNA), or synthetic variants thereof.

24. The method of claim 16, wherein the photoswitchable molecule is an azobenzene, a stilbene, a spiropyran, a fulgide, a diarylethene, a diphenylpolyene, a dihydro-indolizine, a diarylethane, a chromene, a napthopyran, a spiropyran, a fulgide, a fulgimide, a spiroxazine, or any photoswitchable analog thereof.

25. The method of claim 16, wherein the photoswitchable molecule is covalently attached to the first nucleic acid sequence and intercalates between the probe sequence and the target sequence when hybridized.

26. (canceled)

27. The method of claim 16, wherein the photoswitchable molecule is azobenzene, and wherein the first wavelength of light is between about 280 nm and 380 nm.

28. The method of claim 16, wherein at least one of the first nucleic acid and the second nucleic acid is attached to a surface.

29. The method of claim 28, wherein the surface is a particle core that is optically detectable by changes in absorption, light scattering, or photoluminescence that are triggered by changes in the hybridization state of the probe sequence in relation to the target sequence.

30. (canceled)

31. The method of claim 29, wherein the core has a surface plasmon resonance.

32. The method of claim 28, wherein the surface is a planar surface on a substrate.

33. (canceled)

34. The method of claim 16, wherein step (c) further comprises applying a second photonic energy, greater than the first photonic energy, of the first wavelength and monitoring the hybridization state at the first photonic energy and the second photonic energy, and further comprises associating the level of photonic energy at which the conversion to a destabilized, hybridized state or to an unhybridized state between the probe sequence and the target sequence occurs with the photonic energy levels of known base-pair mismatches at the position of the suspected sequence variation, thereby identifying the sequence variation on the target sequence.

35. (canceled)

36. A method for making a probe for detecting a sequence variation in a nucleic acid, comprising:
(a) obtaining the sequence of a reference nucleic acid, or a complement thereof;
(b) determining the location in the reference nucleic acid sequence, or the complement thereof, of a suspected sequence variation;
(c) designating in the reference nucleic acid sequence, or the complement thereof, at least one position within four nucleic acid positions of the location of the suspected sequence variation to receive the incorporation of a photoswitchable molecule, wherein the photoswitchable molecule is capable of undergoing a structural change from a first conformation to a second conformation upon illumination by a first wavelength of light; and
(d) synthesizing a nucleic acid probe that comprises a sequence corresponding to the location in the reference nucleic acid sequence with the suspected sequence variation and a photoswitchable molecule incorporated at the at least one position designated in step (c).


SEQUENCE LISTING (15 sequences)

SEQ ID NO: 1 - DNA, 18 bp, Artificial Sequence
  Synthetic DNA
agactgaaccaatgtatg

SEQ ID NO: 2 - DNA, 18 bp, Artificial Sequence
  Synthetic DNA
tctgacttggttacatac

SEQ ID NO: 3 - DNA, 18 bp, Artificial Sequence
  Synthetic DNA
tctgacttgnttacatac

SEQ ID NO: 4 - DNA, 18 bp, Artificial Sequence
  Synthetic DNA
tctgacttgtttacatac

SEQ ID NO: 5 - DNA, 18 bp, Artificial Sequence
  Synthetic DNA
tctgacttgcttacatac

SEQ ID NO: 6 - DNA, 18 bp, Artificial Sequence
  Synthetic DNA
tctgacttgattacatac

SEQ ID NO: 7 - DNA, 18 bp, Artificial Sequence
  Synethic DNA
tctgacttggctacatac

SEQ ID NO: 8 - DNA, 18 bp, Artificial Sequence
  Synthetic DNA
tctgacttggtgacatac

SEQ ID NO: 9 - DNA, 18 bp, Artificial Sequence
  Synthetic DNA
tctgacttggttgcatac

SEQ ID NO: 10 - DNA, 19 bp, Artificial Sequence
  Synthetic DNA
aaaaaaaaatgaactaacg

SEQ ID NO: 11 - DNA, 37 bp, Artificial Sequence
  Synthetic DNA
caatcatgagcagcctagcagagaagtaaaaaaaaaa

SEQ ID NO: 12 - DNA, 37 bp, Artificial Sequence
  Synthetic DNA
acttgattgcgttagtactcgtcggatcgtctcttca

SEQ ID NO: 13 - DNA, 37 bp, Artificial Sequence
  Synthetic DNA
actttattgcgttagtactcgtcggatcgtctcttca

SEQ ID NO: 14 - DNA, 37 bp, Artificial Sequence
  Synthetic DNA
acttaattgcgttagtactcgtcggatcgtctcttca

SEQ ID NO: 15 - DNA, 37 bp, Artificial Sequence
  Synthetic DNA
acttcattgcgttagtactcgtcggatcgtctcttca